(19) United States
(12) Patent Application Publication
(10) Pub. No.: US 2024/0359025 A1
(43) Pub. Date: Oct. 31, 2024

(54) APPARATUS FOR DELIVERING RADIO FREQUENCY AND CONTROL METHOD THEREOF

(71) Applicant: WONTECH Co., Ltd., Daejeon (KR)

(72) Inventors: Jong Won KIM, Seongnam-si (KR); Jung Hyun KIM, Seongnam-si (KR); Young Seok SEO, Sejong-si (KR)

(21) Appl. No.: 18/348,294

(22) Filed: Jul. 6, 2023

(30) Foreign Application Priority Data
Apr. 27, 2023 (KR) 10-2023-0055303

(51) Int. Cl. A61N 1/40; A61N 1/06

(52) U.S. Cl. CPC A61N 1/40; A61N 1/06

(57) ABSTRACT

Proposed are an apparatus for delivering radio frequency and a control method thereof. The apparatus includes a body configured to control an overall operation, a handpiece connected to the body and configured to be operated by receiving power from the body, and a tip which is attached to a first side surface of the handpiece and which is in close contact with skin of a user. The body includes a first power source unit, a first control unit, a storage unit, an energy generation unit, and a cooling unit. The handpiece includes a second power source unit, a second control unit, an energy transferring unit, and a gas transferring unit. The tip includes a housing, an electrode unit, a data collection unit, and a chamber.

### CROSS REFERENCE TO RELATED APPLICATION

The present application claims priority to Korean Patent Application No. 10-2023-0055303, filed Apr. 27, 2023, the entire contents of which are incorporated herein for all purposes by this reference.

### BACKGROUND OF THE INVENTION

### Field of the Invention

The present disclosure relates to an apparatus for delivering radio frequency. More particularly, the present disclosure relates to an apparatus for delivering radio frequency and a control method of the apparatus configured such that an electrode is brought into close contact with skin and transfers radio frequency under the skin.

### Description of the Related Art

Recently, a technology in which energy is provided to skin by using various energy sources so as to treat the skin by transforming a state of a tissue of the skin or by improving tissue characteristics is widely applied. Skin treatment apparatus using various energy sources such as a laser beam, a flash lamp, an ultrasonic wave, and so on have been developed, and recently, research about a skin treatment apparatus using radio frequency (RF) energy has been actively conducted.

When radio frequency energy is provided to a skin surface, molecules constituting a skin tissue vibrate and rub each other every time a current direction of radio frequency changes, so that deep heat is generated due to rotation, torsion, and collision of the molecules. Such deep heat increases the temperature of the skin tissue and reorganizes the collagen layer, so that wrinkles can be decreased and skin elasticity can be increased. Furthermore, blood circulation in the skin tissue is increased and accelerated, so that overall state of the skin is improved, leading to skin anti-aging.

However, when radio frequency energy is concentrated on the skin, the radio frequency energy may cause damage to the skin or an accident such as burning may occur in a severe case. Therefore, energy emission is required to be controlled properly. When the energy emission is dependent only on a user's operation, a risk of an accident due to the user's negligence or the user's inexperience exists, so that an appropriate control method is required to be provided.

In order to solve the problem described above, a technology of controlling a radio frequency emission time and emitting energy suitable for a resistance value of patient's skin is disclosed. However, due to a heat conduction time of an electrode that is in contact with the skin, there is a problem that a risk of burning is not solved.

### SUMMARY OF THE INVENTION

Accordingly, the present disclosure has been made keeping in mind the above problems occurring in the related art, and an objective of the present disclosure is to provide an apparatus for delivering radio frequency and a control method of the apparatus configured such that wires are placed on both sides of an electrode plate in contact with skin so as to shorten a time to reach a proper temperature, thereby shortening an emission duration time of radio frequency energy and emitting the radio frequency energy several times.

In order to achieve the above objectives, according to the present disclosure, there is provided an apparatus for delivering radio frequency, the apparatus including: a body configured to control an overall operation; a handpiece connected to the body and configured to be operated by receiving power from the body; and a tip which is attached to a first side surface of the handpiece and which is in close contact with skin of a user.

The body may include: a first power source unit coupled to an inside of the body and configured to generate and supply power required for operation; a first control unit coupled to the inside of the body and configured to receive the power of the first power source unit and to perform an operation control; a storage unit which is attached to the first control unit inside the body and which contains data required for the operation control in the first control unit; an energy generation unit attached to the inside of the body and configured to generate radio frequency energy by receiving the power of the first power source unit; and a cooling unit to which a gas can is capable of being attached so that the cooling unit is capable of receiving the power of the first power source unit and cooling gas is capable of being emitted to the skin.

The body may include: a first power source unit coupled to an inside of the body and configured to generate and supply power required for operation; a first control unit coupled to the inside of the body and configured to receive the power of the first power source unit and to perform an operation control; a storage unit which is attached to the first control unit inside the body and which contains data required for the operation control in the first control unit; an energy generation unit attached to the inside of the body and configured to generate radio frequency energy by receiving the power of the first power source unit; and a cooling unit to which a gas can is capable of being attached so that the cooling unit is capable of receiving the power of the first power source unit and cooling gas is capable of being emitted to the skin.

The tip may include: a housing which includes a component of the tip, protects the component from external shock, and includes a coupling apparatus coupled to the handpiece; an electrode unit coupled to an inside of the housing and configured to transfer an electrical signal through an electrical connection with the energy transferring unit, the electrode unit being configured such that the radio frequency energy generated from the energy generation unit of the body is transferred to a first energy applying unit of the handpiece; a data collection unit configured to be operated by receiving power from the electrode unit and configured to measure detailed information of a patient; and a chamber which is formed inside the housing and to which the gas cooling the skin of the user is injected finally through the gas transferring unit of the handpiece when the cooling gas is output from the cooling unit of the body.

The data stored in the storage unit may include data of a radio frequency energy emission time, a cooling gas emission time, a radio frequency energy output amount, and a cooling gas output amount.

The electrode unit of the tip may include: a third power source unit connected to a first side of the second power source unit and configured to receive the power in which the power generated from the first power source unit of the body is transferred through the second power source unit of the handpiece; a first electrode plate and a second electrode plate that are configured to receive the energy from the energy transferring unit in which the energy generated from the energy generation unit of the body is transferred through the energy transferring unit; and a third electrode plate configured to receive the energy from the first electrode plate and the second electrode plate and to be in close contact with the skin of the user and to emit the radio frequency energy.

The cooling gas emission time may be at least longer than the radio frequency energy emission time.

The cooling gas emission time may be 0.1 seconds to 6 seconds.

The cooling gas emission time may be 0.1 seconds to 6 seconds.

In order to achieve the above objectives, according to the present disclosure, there is provided a method of emitting radio frequency, the method including: a process of supplying power to a second power source unit of a handpiece when the power is supplied from a first power source unit of a body; a process of coupling a gas can to a cooling unit of the body and coupling a tip to the handpiece when the power is supplied to the second power source unit of the handpiece; a process of inputting an energy output signal in a first control unit of the body and in a second control unit of the handpiece; a process of closely contacting an electrode unit of the tip to skin of a patient; a process of collecting detailed information of the patient by a data collection unit of the tip when the electrode unit of the tip is in close contact with the skin of the patient; a process of generating a routine to be operated on the patient in the first control unit on the basis of the data collected by the data collection unit; a process of cooling a skin surface by outputting cooling gas from the cooling unit of the body to the skin of the patient on the basis of the routine generated in the first control unit; a process of emitting radio frequency to the electrode unit of the tip through an energy transferring unit of the handpiece when the radio frequency energy is generated in an energy generation unit of the body; a process determining whether the energy output signal is input in the first control unit of the body and in the second control unit of the handpiece; and in the process of determining whether the energy output signal is input in the first control unit and the second control unit, a process of repeating the process of emitting the cooling gas from the cooling unit of the body on the basis of the generated operation routine to the process of determining whether the energy output signal is input in the first control unit of the body and in the second control unit in the handpiece when it is determined that the energy output signal is input.

In the process of collecting the detailed information of the patient by the data collection unit of the tip, the detailed information of the patient collected may be any one of an impedance value, a skin surface temperature, and a skin color value of the patient.

In the process of generating the routine to be operated on the patient in the first control unit on the basis of the data collected by the data collection unit, the routine may be at least one of the number of cooling gas emissions, the number of radio frequency energy emissions, a cooling gas emission time, a radio frequency energy emission time, a cooling gas emission amount, and a radio frequency energy emission amount.

The number of radio frequency energy emission may be 2 to 8 consecutive times.

According to characteristics described above, the present disclosure has an effect that the time to reach the proper temperature of the electrode may be reduced.

In addition, by shortening the emission time for one emitting, there is an effect that burning is prevented compared to a long emission time.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a view illustrating a configuration of an apparatus for delivering radio frequency according to an embodiment of the present disclosure;

FIG. 2 is a schematic view illustrating the apparatus for delivering the radio frequency according to an embodiment of the present disclosure;

FIG. 3 is an exemplary view schematically illustrating a portion of the apparatus for delivering the radio frequency according to an embodiment of the present disclosure;

FIG. 4 is a graph showing a radio frequency energy emission time and a cooling gas emission time of the apparatus for delivering the radio frequency according to an embodiment of the present disclosure; and

FIG. 5 is a flowchart illustrating a control method of the apparatus for delivering the radio frequency according to an embodiment of the present disclosure.

### DETAILED DESCRIPTION OF THE INVENTION

Hereinafter, an embodiment of the present disclosure will be described in detail with reference to the accompanying drawings such that a person having ordinary knowledge in the technical field to which the present disclosure belongs may easily implement the embodiment. It is understood that the present disclosure is able to be implemented in various different forms and is not limited to the embodiment described herein. In addition, parts in the drawings unrelated to the detailed description are omitted to ensure clarity of the present disclosure, and like reference numerals in the drawings denote like elements throughout.

Throughout the specification, when a part is referred to as being “connected” (connect, contact, combine) to another part, it includes being “directly connected” to another part and “indirectly connected” to another part with still another part disposed therebetween. In addition, unless explicitly described to the contrary, the word “comprise” and variations such as “comprises” or “comprising” will be understood to imply the inclusion of stated elements but not the exclusion of any other elements.

The terminology used herein is for the purpose of describing particular embodiments only and is not intended to limit the present disclosure. Singular expressions include plural expressions unless the context clearly indicates otherwise. In the present specification, it is to be understood that terms such as “including”, “having”, etc. are intended to indicate the existence of the features, numbers, steps, actions, elements, parts, or combinations thereof disclosed in the specification, and are not intended to preclude the possibility that one or more other features, numbers, steps, actions, elements, parts, or combinations thereof may exist or may be added.

Then, an apparatus for delivering radio frequency and a control method thereof according to an embodiment of the present disclosure will be described with reference to the accompanying drawings.

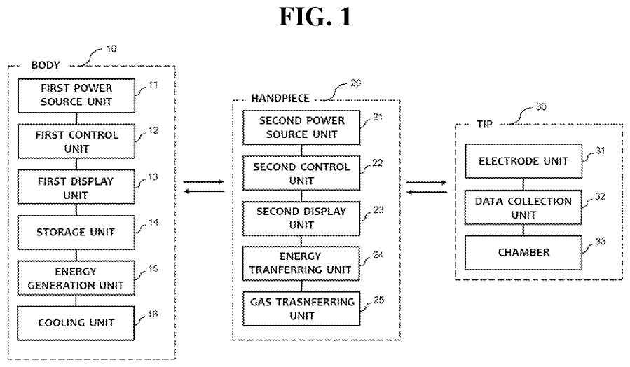

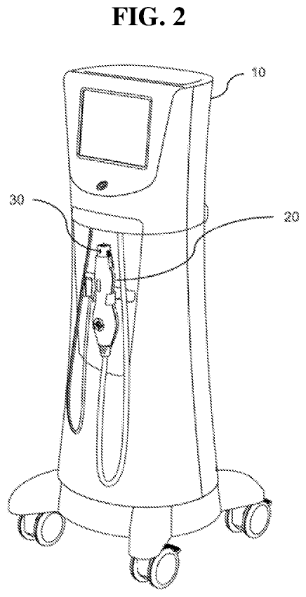

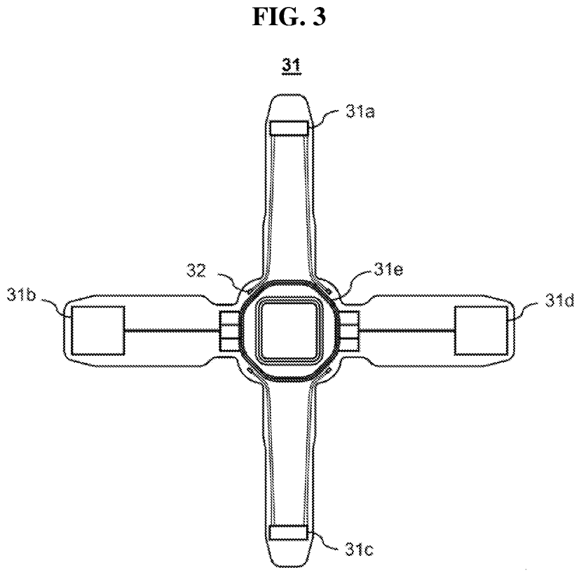

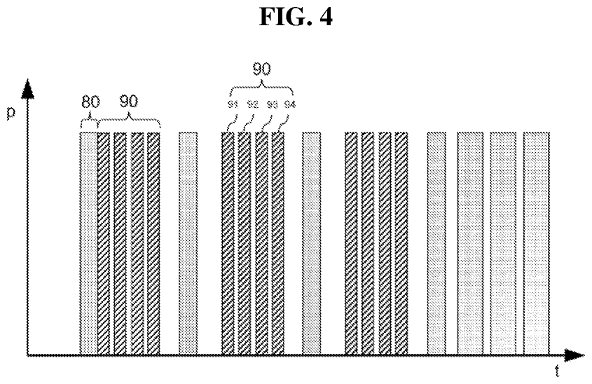

FIG. 1 is a view illustrating a configuration of an apparatus for delivering radio frequency according to an embodiment of the present disclosure, and FIG. 2 is a schematic view illustrating the apparatus for delivering the radio frequency according to an embodiment of the present disclosure. FIG. 3 is an exemplary view schematically illustrating a portion of the apparatus for delivering the radio frequency according to an embodiment of the present disclosure, and FIG. 4 is a graph showing a radio frequency energy emission time and a cooling gas emission time of the apparatus for delivering the radio frequency according to an embodiment of the present disclosure.

Referring to FIGS. 1 to 4, an apparatus for delivering radio frequency includes a body 10 configured to control an overall operation, a handpiece 20 connected to the body 10 and configured to be operated by receiving power from the body 10, and a tip 30 which is attached to a first side surface of the handpiece 20 and which is in close contact with a user's skin.

The body 10 includes a first power source unit 11 coupled to an inside of the body 10 and configured to generate and supply power required for operation, a first control unit 12 coupled to the inside of the body 10 and configured to receive power of the first power source unit 11 and to perform an operation control, a first display unit 13 coupled to a first side of an upper end portion of the body 10 and provided to an operator and a user so as to perform the operation control in the first control unit 12, a storage unit 14 which is attached to the first control unit 12 inside the body 10 and which contains data required for the operation control in the first control unit 12, an energy generation unit 15 attached to the inside of the body 10 and configured to generate radio frequency energy by receiving power of the first power source unit 11, and a cooling unit 16 to which a gas can (not illustrated) is capable of being attached so that the cooling unit 16 is capable of receiving power of the first power source unit 11 and cooling gas is capable of being emitted to the skin.

The handpiece 20 includes a second power source unit 21 coupled to an upper portion of an inner side surface of the handpiece 20 and configured to receive power from the first power source unit 11 of the body 10 in a wired manner and to enable an operation of the handpiece 20, a second control unit 22 fixed to the inner side surface of the handpiece 20, the second control unit 22 having a portion that protrudes in a shape of a plurality of buttons on an outer side surface of the handpiece 20, and the second control unit 22 being configured to be operated by receiving power of the second power source unit 21 and being configured to interwork with the first control unit 12 so that direct control of the user is capable of being performed, a second display unit 23 coupled to the inner side surface of the handpiece 20, the second display unit 23 having a portion exposed to the outside, and the second display unit 23 being configured to be operated by receiving power of the second power source unit 21 and being configured to provide a detailed content of the second control unit 22, an energy transferring unit 24 configured to receive energy generated from the energy generation unit 15 of the body 10 and to transfer the energy to the tip 30, a gas transferring unit 25 for transferring the cooling gas from the gas can of the cooling unit 16 of the body 10, and a casing (not illustrated) including all components described above and protecting the components therein from external shock.

The tip 30 includes a housing which includes a component of the tip 30, protects the component from external shock, and includes a coupling apparatus (not illustrated) coupled to the handpiece 20, an electrode unit 31 coupled to an inside of the housing and configured to transfer power through an electrical connection with the second power source unit 21 of the handpiece 20 and to transfer an electrical signal through an electrical connection with the energy transferring unit 24, the electrode unit 31 being configured such that radio frequency energy generated from the energy generation unit 15 of the body 10 is transferred to a first energy applying portion of the handpiece 20 and is finally emitted to the user's skin, a data collection unit 32 configured to be operated by receiving power from the electrode unit 31 and configured to measure detailed information of a patient, and a chamber 33 which is formed inside the housing and to which gas cooling the user's skin is injected finally through the gas transferring unit 25 of the handpiece 20 when the cooling gas is output from the cooling unit 16 of the body 10.

The first display unit 13 provides a control UI to the operator, and the first control unit 12 is operated by a detailed control of the operator.

The storage unit 14 includes data about a detailed setting required for operation in the first control unit 12.

The data stored in the storage unit 14 includes data of a radio frequency energy emission time, a cooling gas emission time, a radio frequency energy output amount, and a cooling gas output amount, and the data is not limited thereto.

The cooling unit 16 further includes a casing (not illustrated) to which a cooling gas can (not illustrated) is attached, a gas output unit for discharging gas, the gas output unit being formed on a distal end of the casing, and a connection pipe (not illustrated) connected to a first side of the gas output unit and configured to transfer the cooling gas by being connected to a distal end of the gas transferring unit 25 of the handpiece 20 when the gas is emitted from the gas output unit.

The second control unit 22 of the handpiece 20 has a shape which is inserted into a hole (not illustrated) perforating the casing of the handpiece 20 and which protrudes, or has a touchpad shape.

The second display unit 23 of the handpiece 20 displays an operation state to the user and the patient according to an input value of the first control unit 12 of the body 10 and an input value of the second control unit 22 of the handpiece 20.

The gas transferring unit 25 of the handpiece 20 is connected to a first side of the connection pipe of the cooling unit 16 of the body 10, and is configured to transfer the cooling gas to the chamber 33 of the tip 30 when the gas is discharged from the cooling unit 16.

Detailed information of the patient collected in the data collection unit 32 is any one of an impedance value of the patient, a skin surface temperature of the patient, and a skin color value of the patient, but is not limited thereto.

The electrode unit 31 of the tip 30 includes third power source units 31a and 31c connected to a first side of the second power source unit 21 and configured to receive power in which the power generated from the first power source unit 11 of the body 10 is transferred through the second power source unit 21 of the handpiece 20, a first electrode plate 31b and a second electrode plate 31d that are configured to receive energy from the energy transferring unit 24 in which the energy generated from the energy generation unit 15 of the body 10 is transferred through the energy transferring unit 24, and a third electrode plate 31e configured to receive the energy from the first electrode plate 31b and the second electrode plate 31d and to be in close contact with the user's skin and to emit radio frequency energy.

By using the configuration of the tip 30 as described above, energy is simultaneously transferred to the third electrode plate 31e from the first electrode plate 31b and the second electrode plate 31d, so that there is an effect that a time to reach a proper temperature is shortened.

Referring to the graph in FIG. 4, the x-axis and the y-axis represent an emission time (t) and an emission amount (p) for a cooling gas emission 80 and a radio frequency energy emission 90.

The cooling gas emission 80 time is at least longer than the radio frequency emission 90 time. More specifically, the cooling gas emission 80 time is 0.1 to 6 seconds and the radio frequency emission 90 time is 0.2 to 7 seconds, but are not limited thereto.

Then, a method of emitting radio frequency according to an embodiment of the present disclosure will be described with reference to the accompanying drawings.

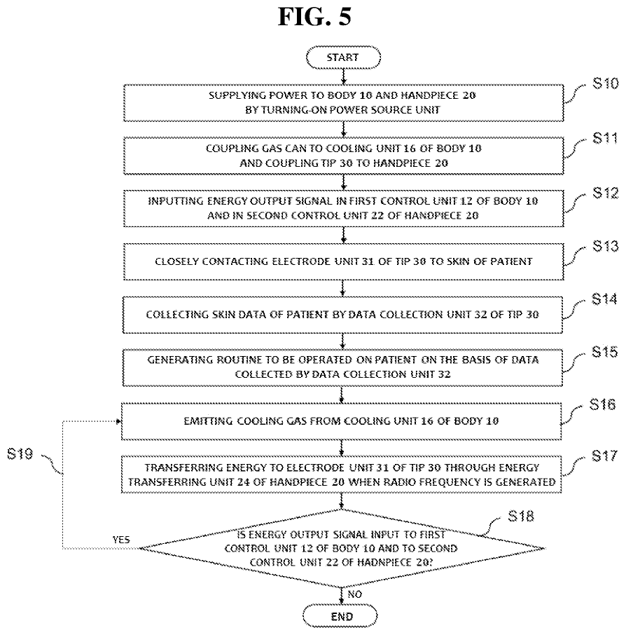

FIG. 5 is a flowchart illustrating a control method of the apparatus for delivering the radio frequency according to an embodiment of the present disclosure.

Referring to FIG. 5, first, when power is supplied from the first power source unit 11 of the body 10, the power is supplied to the second power source unit 21 of the handpiece 20 S10.

When the power is supplied to the second power source unit 21 of the handpiece 20, a gas can is coupled the cooling unit 16 of the body 10, and the tip 30 is coupled to the handpiece 20 S11.

An energy output signal is input from the first control unit 12 of the body 10 and the second control unit 22 of the handpiece 20 S12.

The electrode unit 31 of the tip 30 is brought into close contact with a patient's skin S13.

When the electrode unit 31 of the tip 30 is in close contact with the patient's skin, the patient's detailed information is collected by the data collection unit 32 of the tip 30 S14.

At this time, the patient's detailed information collected is any one of a patient's impedance value, a patient's skin surface temperature, and a patient's skin color value, but is not limited thereto.

A routine to be operated on the patient is generated in the first control unit 12 on the basis of the data collected by the data collection unit 32 S15.

At this time, the routine is different for each user, and is at least one of the number of cooling gas emissions, the number of radio frequency energy emissions, a cooling gas emission time, a radio frequency energy emission time, a cooling gas emission amount, and a radio frequency energy emission amount, but is not limited thereto.

In addition, in the data collected by the data collection unit 32, when data about a patient's skin temperature is higher than a preset temperature, the number of cooling gas emissions may be further increased before emitting radio frequency energy is started.

Firstly, on the basis of the routine generated in the first control unit 12, the cooling gas is output to the patient's skin from the cooling unit 16 of the body 10 and the skin surface is cooled S16.

Next, when radio frequency energy is generated from the energy generation unit 15 of the body 10, the radio frequency energy is emitted to the electrode unit 31 of the tip 30 through the energy transferring unit 24 of the handpiece 30 S17.

At this time, the cooling gas emission time is at least longer than the radio frequency energy emission time, the cooling gas emission time is 0.1 to 6 seconds, the radio frequency energy emission time is 0.2 to 7 seconds, and are not limited thereto.

In addition, the number of radio frequency energy emissions is 2 to 8 consecutive times, but is not limited thereto.

Next, whether an energy output signal is input from the first control unit 12 of the body 10 and the second control unit 22 of the handpiece 20 is determined S18.

In the process of determining whether the energy output signal is input from the first control unit 12 and the second control unit 22, when it is determined that the energy output signal is input, the process of emitting the cooling gas from the cooling unit 16 of the body 10 on the basis of the generated operation routine S16 to the process of determining whether the energy output signal is input from the first control unit 12 of the body 10 and the second control unit 22 of the handpiece 20 S18 are repeated.

In the process of determining whether the energy output signal is input from the first control unit 12 and the second control unit 22, when it is determined that the energy output signal is not input determined to be not input, a procedure is finished.

Although the embodiments of the present disclosure have been described above in detail, the scope of right of the present disclosure is not limited thereto, and various changes and modifications by those skilled in the art using the basic concept of the present disclosure defined in the following claims are also included in the scope of right of the present disclosure.

What is claimed is:

1. An apparatus for delivering radio frequency, the apparatus comprising:
a body configured to control an overall operation;
a handpiece connected to the body and configured to be operated by receiving power from the body; and
a tip which is attached to a first side surface of the handpiece and which is in close contact with skin of a user, and
wherein the body comprises:
a first power source unit coupled to an inside of the body and configured to generate and supply power required for operation;
a first control unit coupled to the inside of the body and configured to receive the power of the first power source unit and to perform an operation control;
a storage unit which is attached to the first control unit inside the body and which contains data required for the operation control in the first control unit;
an energy generation unit attached to the inside of the body and configured to generate radio frequency energy by receiving the power of the first power source unit; and
a cooling unit to which a gas can is capable of being attached so that the cooling unit is capable of receiving the power of the first power source unit and emitting cooling gas to the skin,
the handpiece comprises:
a second power source unit coupled to an upper portion of an inner side surface of the handpiece and configured to receive the power from the first power source unit of the body in a wired manner and to enable an operation of the handpiece;
a second control unit fixed to the inner side surface of the handpiece, the second control unit having a portion that protrudes in a shape of a plurality of buttons on an outer side surface of the handpiece, and the second control unit being configured to be operated by receiving power of the second power source unit and being configured to interwork with the first control unit so that direct control of the user is capable of being performed;
an energy transferring unit configured to receive energy generated from the energy generation unit of the body and to transfer the energy to the tip; and
a gas transferring unit for transferring the cooling gas from the gas can of the cooling unit of the body, and
the tip comprises:
a housing which comprises a component of the tip, protects the component from external shock, and comprises a coupling apparatus coupled to the handpiece;
an electrode unit coupled to an inside of the housing and configured to transfer an electrical signal through an electrical connection with the energy transferring unit, the electrode unit being configured such that the radio frequency energy generated from the energy generation unit of the body is transferred to a first energy applying portion of the handpiece;
a data collection unit configured to be operated by receiving power from the electrode unit and configured to measure detailed information of a patient; and
a chamber which is formed inside the housing and to which the gas cooling the skin of the user is injected finally through the gas transferring unit of the handpiece when the cooling gas is output from the cooling unit of the body.

2. The apparatus of claim 1, wherein the data stored in the storage unit comprises data of a radio frequency energy emission time, a cooling gas emission time, a radio frequency energy output amount, and a cooling gas output amount.

3. The apparatus of claim 1, wherein the electrode unit of the tip comprises:
a third power source unit connected to a first side of the second power source unit and configured to receive power from the second power source unit, the power generated from the first power source unit of the body being transferred to the third power source unit through the second power source unit of the handpiece;
a first electrode plate and a second electrode plate that are configured to receive energy from the energy transferring unit, the energy generated from the energy generation unit of the body being transferred to the first electrode plate and the second electrode plate through the energy transferring unit; and
a third electrode plate configured to receive the energy from the first electrode plate and the second electrode plate and to be in close contact with the skin of the user and to emit the radio frequency energy.

4. The apparatus of claim 2, wherein the cooling gas emission time is at least longer than the radio frequency energy emission time.

5. The apparatus of claim 2, wherein the cooling gas emission time is between 0.1 seconds to 6 seconds.

6. The apparatus of claim 2, wherein the radio frequency energy emission time is between 0.2 seconds to 7 seconds.

7. A method of emitting radio frequency, the method comprising:
a process of supplying power to a second power source unit of a handpiece when the power is supplied from a first power source unit of a body;
a process of coupling a gas can to a cooling unit of the body and coupling a tip to the handpiece when the power is supplied to the second power source unit of the handpiece;
a process of inputting an energy output signal in a first control unit of the body and in a second control unit of the handpiece;
a process of closely contacting an electrode unit of the tip to skin of a patient;
a process of collecting detailed information of the patient by a data collection unit of the tip when the electrode unit of the tip is in close contact with the skin of the patient;
a process of generating a routine to be operated on the patient in the first control unit on the basis of the data collected by the data collection unit;
a process of cooling a skin surface by outputting cooling gas from the cooling unit of the body to the skin of the patient on the basis of the routine generated in the first control unit;
a process of emitting radio frequency to the electrode unit of the tip through an energy transferring unit of the handpiece when the radio frequency energy is generated in an energy generation unit of the body;
a process of determining whether the energy output signal is input in the first control unit of the body and in the second control unit of the handpiece; and
in the process of determining whether the energy output signal is input in the first control unit and the second control unit, a process of repeating the process of emitting the cooling gas from the cooling unit of the body on the basis of the generated operation routine to the process of determining whether the energy output signal is input in the first control unit of the body and in the second control unit in the handpiece when it is determined that the energy output signal is input.

8. The method of claim 7, wherein, in the process of collecting the detailed information of the patient by the data collection unit of the tip, the detailed information of the patient collected is any one of an impedance value, a skin surface temperature, and a skin color value of the patient.

9. The method of claim 7, wherein, in the process of generating the routine to be operated on the patient in the first control unit on the basis of the data collected by the data collection unit, the routine is at least one of the number of cooling gas emissions, the number of radio frequency energy emissions, a cooling gas emission time, a radio frequency energy emission time, a cooling gas emission amount, and a radio frequency energy emission amount.

10. The method of claim 9, wherein the number of radio frequency energy emission is 2 to 8 consecutive times.
